(19) United States
(12) Patent Application Publication
(10) Pub. No.: US 2009/0048649 A1
(43) Pub. Date: Feb. 19, 2009

(54) Heat transfer device: seal and thermal energy contact units

(75) Inventors: James Peret, Boylston, MA (US); Laura C. Grisanti, West Seneca, NY (US); Gerard E. Kedge, Orchard Park, NY (US); Karl Cazzini, Orchard Park, NY (US); James McGee, Watertown, MA (US)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: Gaymar Industries, Inc.

(21) Appl. No.: 11/893,446

(22) Filed: Aug. 16, 2007

(51) Int. Cl. A61F 7/00

(52) U.S. Cl. 607/100; 607/104; 607/108

(57) ABSTRACT

A device has four fundamental components: an enclosure, a soft seal, a vacuum system and a thermal energy system having a thermal energy contacting element. The device provides thermal energy therapy and negative pressure therapy device simultaneously and/or in conjunction to a patient. The device has a thermal energy system that is more efficient in thermal energy transfer and the soft seal decreases tissue interface pressure to obtain the desired soft seal effect.

### FIELD OF THE INVENTION

The present invention relates to negative pressure, thermal energy transfer units for controlling, maintaining and/or adjusting the body core temperature of a mammal, in particular a human.

### BACKGROUND OF THE INVENTION

Stanford University is the assignee of U.S. Pat. Nos. 5,683,438; 6,602,277; 6,673,099; 6,656,208; 6,966,922; 7,122,047; and 6,974,442. These patents disclose a negative pressure, thermal energy device that can be applied to a patient. The negative pressure device has the following elements: (1) an enclosure having an opening to receive a portion of a patient's body that contains a non-hairy skin region overlaying the subcutaneous arteriovenous anastomoses (AVAs) and venous plexuses (henceforth the non-hairy skin region and underlying area is collectively referred to as a “venous plexus area”), (2) a vacuum system that creates a negative pressure in the enclosure, (3) a seal positioned at the enclosure's opening to maintain the negative pressure in the enclosure, and (4) a thermal energy system having a thermal energy contacting element wherein the venous plexus area is exposed to the thermal energy from the thermal energy contacting element.

### Enclosure

The enclosure surrounds a portion of a patient's body. Hypothetically the enclosure could surround any portion of the patient's body. In a preferred embodiment, however, the portion of the patient's body has a venous plexus area. The venous plexus area has a vascular network formed by numerous anastomoses between veins. A venous plexus area is normally located at the patient's foot area and/or hand area.

The enclosure can be shaped like a glove, a mitten, a boot, a clam-shell, or equivalents thereof so long as there is an opening that can receive the patient's body part. In many embodiments, the enclosure is a polymeric material that can withstand the formation of predetermined negative pressure values within its interior that receives the patient's body part, normally having a venous plexus area.

### Seal

The seal is mounted at the enclosure's opening that receives the patient's body part having a venous plexus area. The seal establishes (1) a vacuum-tight fit between the body portion and the enclosure or (2) a soft seal fit between the body portion and the enclosure.

The term “vacuum-tight”, as interpreted by Dr. Grahn in some of the above-identified Stanford patents and he is one of the inventors of all of the Stanford patents, means a hard seal. In U.S. Pat. No. 7,182,776; Dr. Grahn wrote, “A “hard” seal is characterized as one designed to altogether avoid air leakage past the boundary it provides. In theory, a “hard” seal will allow a single evacuation of the negative pressure chamber for use in the methods. In practice, however, a “hard” seal can produce a tourniquet effect. Also, any inability to maintain a complete seal will be problematic in a system requiring as much.”

A “soft” seal as described herein is characterized as providing an approximate or imperfect seal at a user/seal interface. Such a seal may be more compliant in its interface with a user. Indeed, in response to user movement, such a seal may leak or pass some air at the user/seal interface. In a negative-pressure system designed for use with a soft seal, a regulator or another feedback mechanism/routine will cause a vacuum pump, generator, fan or any such other mechanism capable of drawing a vacuum to respond and evacuate such air as necessary to stabilize the pressure within the chamber, returning it to the desired level. Active control of vacuum pressure in real-time or at predetermined intervals in conjunction with a “soft” seal provides a significant advantage over a “hard” seal system that relies on simply pulling a vacuum with the hopes of maintaining the same.

Some of the Stanford patents disclose the seal is long to “provide greater seal surface contact with a user.” Greater seal surface contact to the patient increases tissue interface pressure. Increased tissue interface pressure is undesirable.

The present invention is designed to address this issue.

### Vacuum System

The vacuum system connects to the enclosure for generating and, in some embodiments, maintaining a predetermined negative pressure inside the enclosure to cause, in conjunction with the other components of the negative pressure, thermal energy device, vasodilation in the body portion surrounded in the enclosure. Negative pressure conditions are a pressure lower than ambient pressure under the particular conditions in which the method is performed. The magnitude of the decrease in pressure from the ambient pressure under the negative pressure conditions is generally at least about 20 mmHg, usually at least about 30 mmHg and more usually at least about 35 mmHg, where the magnitude of the decrease may be as great as 85 mmHg or greater, but typically does not exceed about 60 mmHg and usually does not exceed about 50 mmHg. Applying the negative pressure condition to a portion of the body in the enclosure (a) lowers the vasoconstriction temperature and/or (b) increases vasodilation in the body portion that is in the enclosure.

The negative pressure inducing element may be actuated in a number of different ways, including through motor driven aspiration, through a system of valves and pumps which are moved through movement of the mammal in a manner sufficient to create negative pressure in the sealed environment, etc.

### Thermal Energy Contacting Element

The thermal energy contacting element transfers thermal energy to, or extracts thermal energy from the body portion in the vacuum enclosure. Whether the thermal energy transfers to or extracts from the body portion depends on the relative temperatures of the thermal energy contacting element and the body portion. The vasodilation in the body portion enhances the exchange of thermal energy between a patient's body core, surface of the body portion, and the thermal energy contacting element.

The thermal energy contacting element has been disclosed as (a) “a radiant heat lamp” (i) positioned exterior to the enclosure and (ii) that provides radiant heat to the exterior surface of the enclosure which warms the interior of the enclosure and thereby provides warm thermal energy to the body portion in the enclosure—not just a specific portion of the body portion in the enclosure, (b) warming or cooling blankets, warm or cool water immersion elements, warming or cooling gas elements, a curved metal plate or a metal tube positioned in the interior of the enclosure. The latter embodiments can have a fluid (i) circulate within it and (ii) not contact the body portion in the desired area—the venous plexus area.

In relation to the non-radiant embodiments, a patient could elect (a) not to grip the thermal energy contacting element, (b) to re-position the body part, so the body part is not affected by the thermal energy contacting element or (c) to loosen (for example blanket embodiments) the thermal energy contacting element so it does not effectively contact the body part. The patient's election may be unintentional especially if the patient is sedated or under general anesthesia. It is therefore at least one object of the present invention to solve this potential gripping problem especially for venous plexus areas by making the device invariant to a patient's desire to “grip” the thermal energy contacting element.

Of these thermal energy contacting element embodiments, the metal plate and tube are considered by at least some of the inventors to be the most effective thermal energy contacting elements because (a) those components are easy to manufacture, (b) the thermal energy transfer efficiency to the patient is relatively acceptable and (c) the ease of using the product in actual use.

The curved metal plate and/or the metal tube are shaped to receive a conventional hand and/or foot. Unlike machined products, there is no standard sized hand or foot. That means the energy transfer efficiency for current thermal energy contacting elements may not be maximized for maximum contact with a patient's venous plexus area.

One embodiment of the current invention decreases the tissue interface pressure which causes injuries to the patient's skin. One of those injuries is the equivalent to a bruise that is found with patient's having bed sores. Obviously a negative pressure, thermal energy transfer device that increases tissue interface pressure is undesirable.

The fluid temperature can be thermally controlled and delivered to the thermal energy contacting element by, for example, Gaymar's Medi-Therm III fluid thermal control dispensing unit.

### SUMMARY OF THE INVENTION

A body core temperature control device has four fundamental components: an enclosure, a soft seal, a vacuum system and a thermal energy system having a thermal energy contacting element. The device provides thermal energy therapy and negative pressure therapy simultaneously and/or in conjunction to a patient. The device has a thermal energy system that is more efficient in thermal energy transfer to the body core and the soft seal decreases tissue interface pressure to obtain the desired soft seal effect.

The improvements to the soft seal and the thermal energy transfer device, and some modifications of the enclosure, are significant improvements over the prior art, and effectively obtain the desired goal of controlling the body core temperature.

### BRIEF DESCRIPTION OF THE FIGURES

FIGS. 1 to 9 illustrate nine embodiments of a negative pressure, thermal energy device that can be interchanged with each other to obtain the desired maximum efficiency of the negative pressure, thermal energy device.

FIG. 10 illustrates the crown structure of the foam material used in FIG. 9.

### DETAILED DESCRIPTION OF THE INVENTION

The present invention is a negative pressure, thermal energy device 10 having an enclosure 12, a seal 14, a vacuum system 16 and a thermal energy system 18 having a thermal energy contacting element 20.

It is to be understood that if a fluid circulates through the thermal energy contacting element 20 the fluid comes from the thermal energy system 18. The thermal energy system 18 can have a component that delivers a fluid having a predetermined temperature. An example of such a component is Gaymar's Medi-Therm delivery and thermal control unit. It is also understood that if electricity is required for thermal energy contacting element 20 (if a resistor system is used) that the thermal energy system has an electrical wire interconnected to an electrical providing source, like an electrical outlet.

The vacuum system 16 and the thermal energy system 18 (excluding the thermal energy contacting element 20) are identical and/or similar to the prior art. The vacuum system provides the desired negative pressure within the enclosure 12 through conduit 16a. Likewise, thermal energy system 18 provides the desired thermal energy to the thermal energy contacting element 20 through a conduit 18a (electrical and/or fluid conduit.) As such we will not describe those general features of the thermal energy system 18 or the vacuum system 16 in great detail in this portion of the application. There are, however, a few changes to these systems 16, 18 that are disclosed below.

The enclosure 12 remains fundamentally the same as the enclosure 12 disclosed in the prior art with some exceptions. Those exceptions are disclosed in greater detail below.

Set forth below are examples of numerous embodiments of the present invention. These embodiments can, in some instances, be interchanged with each other to obtain the desired maximum efficiency of energy transfer and decrease the potential damage to the patient's skin.

### First Embodiment

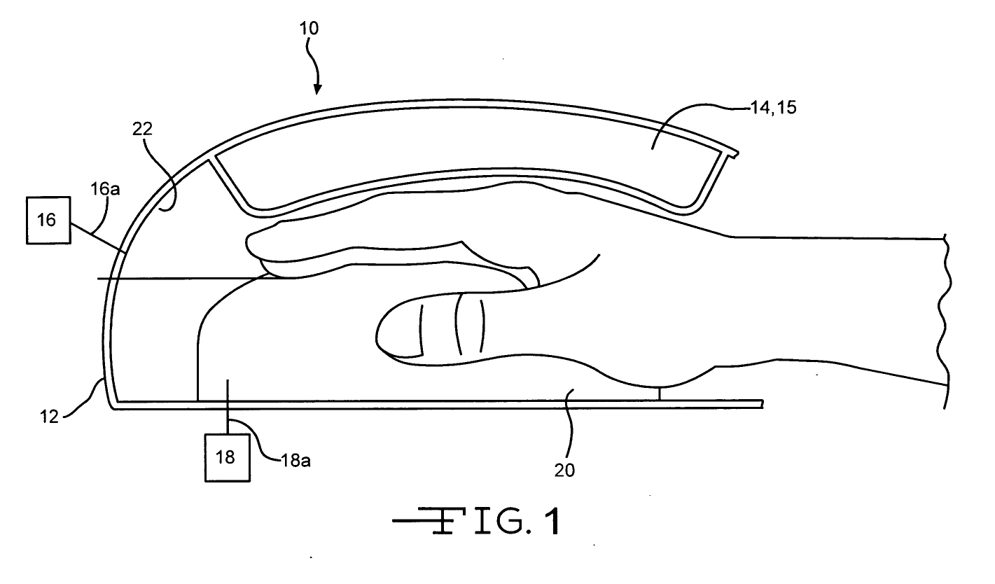

A first embodiment of the negative pressure, thermal energy device 10 is illustrated in FIG. 1. In FIG. 1, the thermal energy contacting element 20 has a symmetric design to accommodate both small and large hands, and can have a fluid and/or a resistor system. The seal is a critical element in this embodiment.

The seal 14 is a bladder seal 15. The bladder seal 15 and the thermal energy contacting element 20 are positioned in the interior of the enclosure 12. In particular the bladder seal 15 and the thermal energy contacting element 20 are positioned on the opposite sides of the interior surface 22 of the enclosure 12, as illustrated in FIG. 1. The bladder seal 15 receives a fluid at a predetermined pressure. Preferably the predetermined pressure in the bladder seal 15 is around 32 mmHg.

The bladder seal 15 inflates to the predetermined pressure when the patient's venous plexus area is properly positioned on the thermal energy contacting element 20.

The fluid in the bladder seal 15 can be air, water, or any other fluid that can be provided to the bladder seal at the predetermined pressure. In one embodiment the fluid can be provided by Gaymar's Medi-Therm device that could be used with the thermal energy system 18. It is known that two of Gaymar's Medi-Therm devices can deliver two fluids (same or different) having two different (or same) temperatures to two different locations at the same time at the same or different pressures.

The bladder seal 15 allows the negative pressure in the enclosure 12 to leak to create the desired soft seal. The bladder seal 15 simultaneously applies some insignificant pressure to the opposite side of the patient's venous plexus area to merely ensure the patient's venous plexus area contacts the thermal energy contacting element 20. The back side pressure ensures the patient's body portion is properly contacting the thermal energy contacting element 20. The back side pressure is also sufficient not to (a) cause tissue damage to the patient and (b) inhibit the blood flow through the venous plexus area.

When the patient's body portion is to be removed from the negative pressure, thermal energy device 10, the bladder seal 15 is deflated (or partially deflated) to allow the patient's body portion to be easily removed with minimal effort.

By applying the desired back side pressure, the patient is unable to manipulate its body portion to avoid the desired maximum efficiency of transferring thermal energy from the thermal energy contacting element 20 to the body portion.

### Second Embodiment

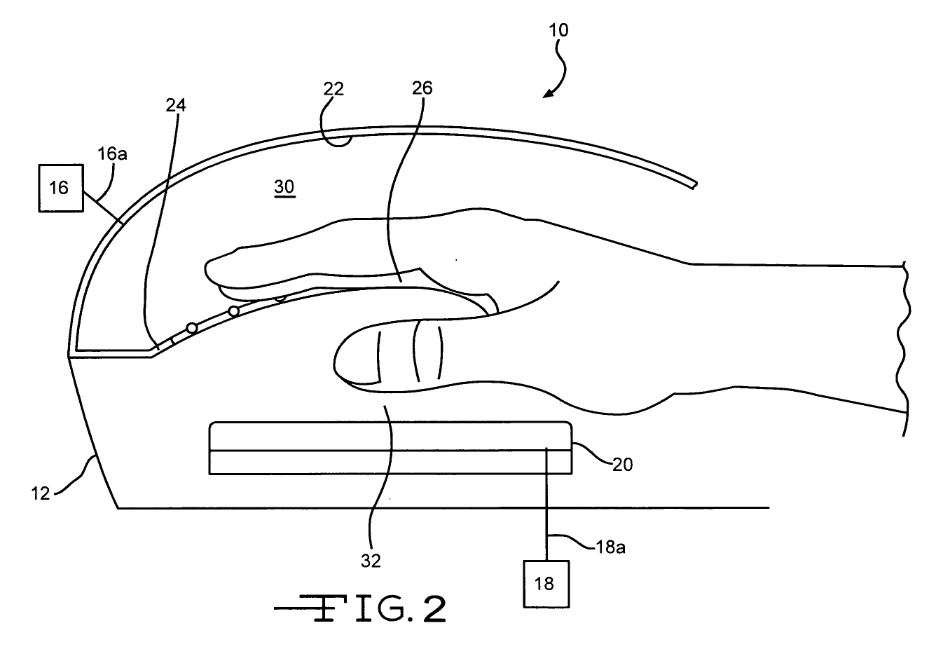

FIG. 2 illustrates variations of the enclosure 12 and the thermal energy contacting element 20. The enclosure 12 has the interior surface 22, and extending from the interior surface 22 is a mezzanine layer 24.

The mezzanine layer 24 divides the enclosure's 12 interior into two sections. The first section 30 receives the patient's body portion. The second section 32 contains the thermal energy contacting element 20. The thermal energy contacting element 20 can be (a) a light source that radiates heat having a narrow or a broad band of non-ionizing light (500 to 2000 nm), or (b) a cold source, for example, dry ice, that extracts thermal energy from the patient's venous plexus area.

The mezzanine layer 24 is a material that allows the radiant thermal energy from (a) the thermal energy contacting element 20 to pass through it or (b) the patient's venous plexus area to pass through it.

The light source embodiment, on first blush, may seem similar to the prior art's heat lamp embodiment but it is not.

As illustrated in FIG. 2, the present invention has the thermal energy contacting element 20 positioned (i) within the enclosure 12 and (ii) to direct thermal energy toward the patient's body portion's venous plexus area. That way the thermal energy from the thermal energy contacting element 20 is directed toward the patient's venous plexus area, and the majority of other portions of the patient's body contained within the enclosure 12 are not directly exposed to the radiant energy.

To inhibit the chances that the patient's venous plexus area is damaged by the thermal energy, in particular the radiant energy, the mezzanine layer 24 is positioned a predetermined distance from the thermal energy contacting element 20. In addition the mezzanine layer 24 can have thermocouples 26 embedded or attached thereto. The thermocouples 26 measure the thermal energy applied to the patient's venous plexus area. The thermocouple 26 transmits a measurement signal of the thermal energy measurement and transmits that measurement to the thermal energy system 18. Depending on the measurement signal in relation to a desired thermal energy temperature to be applied to the patient's venous plexus area, the thermal energy system 18 can alter the radiant thermal energy level generated from the thermal energy contacting element 20 to obtain the desired thermal energy temperature to be applied to the patient's venous plexus area.

Any seal 14 (not shown in FIG. 2) described in the current section entitled detailed description of the invention, and the prior art (though undesirable), can be used with this second embodiment.

### Third Embodiment

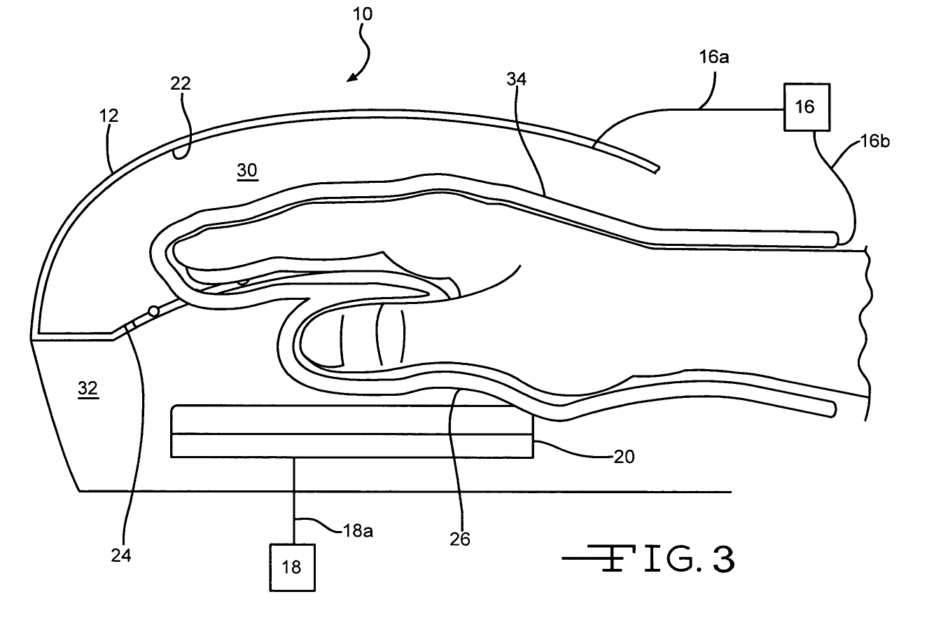

The third embodiment is illustrated in FIG. 3. The third embodiment is similar to the second embodiment. A difference between the third and the second embodiments is that the thermal sensors 26 are embedded and/or attached to a fitting material 34 (for example, a glove, a mitten, and/or a sock) positioned over the patient's body portion that enters the enclosure 12. In particular the thermal sensors 26 are positioned next to the patient's venous plexus area as illustrated in FIG. 3.

Preferably, the first fitting material 34 is a black material to retain the thermal energy transferred to the patient.

In addition, a slight negative pressure from the vacuum system 16 though conduit 16b could be applied to the interior portion of the first fitting material 34. The conduit 16b could have, or not have, valves and check valves to ensure the pressure in the first fitting material 34 is the same or different from the negative pressure in the first section 30 of the enclosure 12. That slight negative pressure provides the desired contact between the patient's body portion and the first fitting material 34. As such, the efficiency of the transfer of thermal energy from the thermal energy contacting element 20 to the patient's body portion is maximized.

Any seal 14 (not shown in FIG. 3) described in the current section entitled detailed description of the invention, and the prior art (though undesirable), can be used with this third embodiment.

The fitting material should provide sufficient pressure so it does not inhibit the blood flow through the venous plexus area but assists in the creation of the desired vasodilation that is required.

### Fourth Embodiment

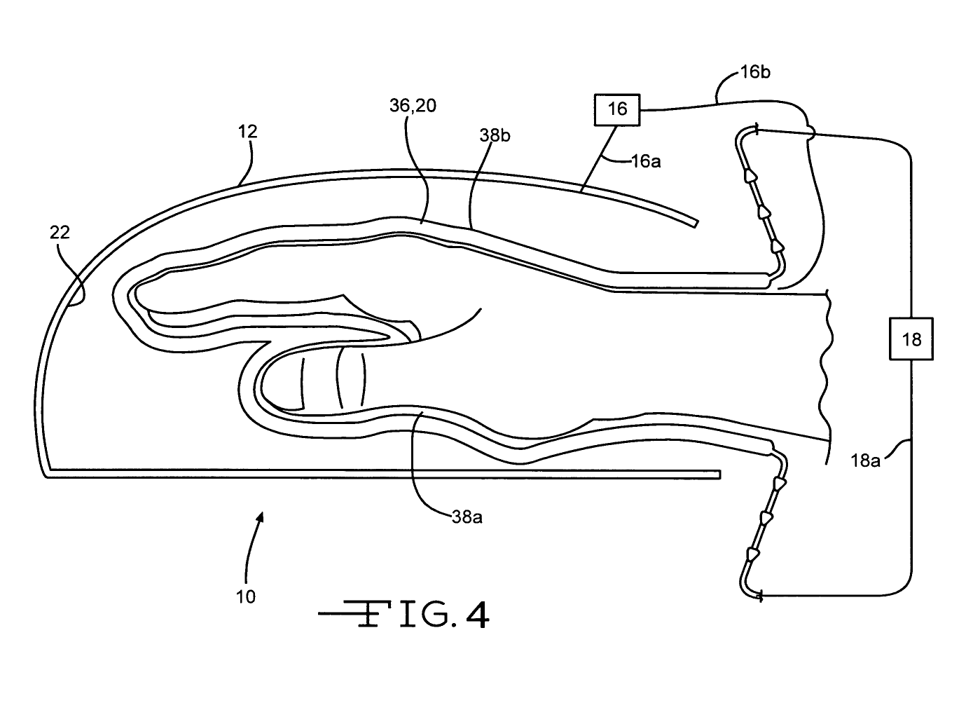

A fourth embodiment of the present invention is illustrated in FIG. 4. The fourth embodiment uses a convective fitting object 36 as the thermal energy contacting element 20. The convective fitting object 36 is a glove, a mitten, a blanket and/or a sock, positioned over the patient's body portion. As with any convective fitting object 36, the object has an interior layer 38a interconnected to an exterior layer 38b. The exterior layer 38b and the interior layer 38a can be a single piece of material, different pieces of material, same type of materials and/or different types of materials. The only requirement is that the interior layer 38a does not allow the fluid that circulates between the interior layer 38a and the exterior layer 38b to contact the patient's body portion. Also, the exterior layer 38b should not allow the fluid that circulates between the interior layer 38a and the exterior layer 38b to contact the enclosure 12.

In addition, a slight negative pressure from the vacuum system 16 could be applied between the patient's body portion and the interior layer 38a of the convective fitting object 36. That slight negative pressure provides the desired contact between the patient's body portion and the first fitting material 34. As such, the efficiency of the transfer of thermal energy from the thermal energy contacting element 20 to the patient's body portion is maximized.

Any seal 14 (not shown in FIG. 4) described in the current section entitled detailed description of the invention, and the prior art (though undesirable), can be used with this fourth embodiment.

The fitting material and the negative pressure applied between the interior layer 38a and the patient should provide sufficient pressure and not inhibit the blood flow through the venous plexus area but assists in the creation of the desired vasodilation that is required.

### Fifth Embodiment

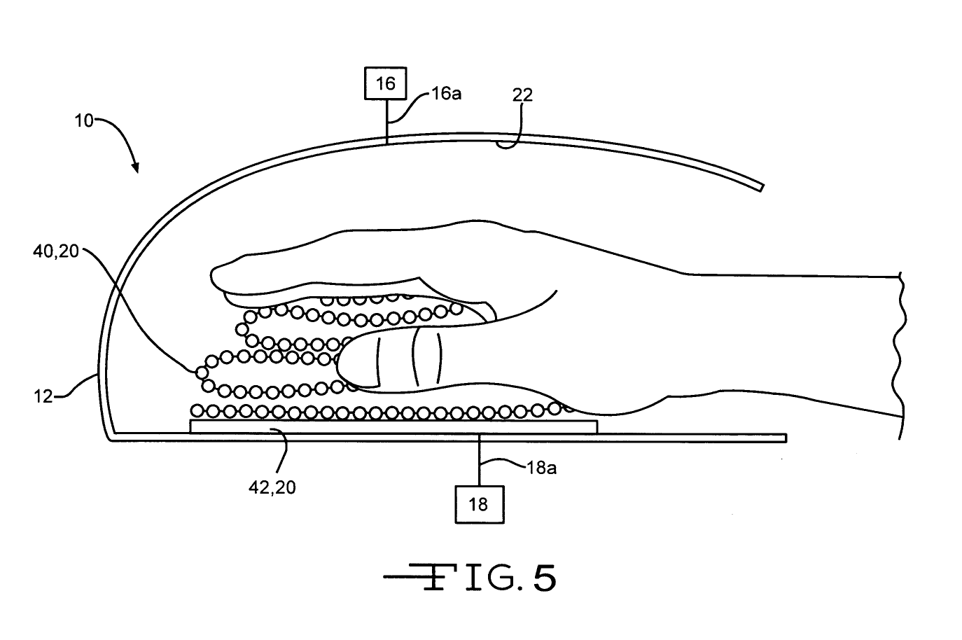

FIG. 5 illustrates a fifth embodiment of the present invention. In this embodiment the thermal energy contacting element 20 is conductive beads 40 (for example, polymeric with metal material, or metal beads interconnected by a conductive wire) extending from a conductive plate 42. The conductive plate 42 can be planar as illustrated in FIG. 5, a curved metal piece, radiant energy source as illustrated in FIGS. 2 and 3, or a symmetric design as illustrated in FIG. 1. The conductive plate 42 just has to be able to transfer some of its thermal energy from to the conductive beads 40. The conductive beads 40 increase the surface area of the thermal energy contacting element 20 and allow for conformability of the patient's body portions—in particular the venous plexus area. As such, the efficiency of the transfer of thermal energy from the thermal energy contacting element 20 to the patient's body portion is maximized.

Any seal 14 (not shown in FIG. 5) described in the current section entitled detailed description of the invention, and the prior art (though undesirable), can be used with this fifth embodiment.

The enclosure 12 and the vacuum system 16 can be the conventional embodiments for this fifth embodiment.

### Sixth Embodiment

The sixth figure illustrates a sixth embodiment of the negative pressure, thermal energy device 10. The enclosure 12 has the interior surface 22, and extending from the interior surface is a second mezzanine layer 24a.

The second mezzanine layer 24a separates the interior of the enclosure 12 into two sections. The first section 50 receives the patient's body portion. The second section 52 only contains a portion of the thermal energy contacting element 20. The second mezzanine layer 24a also positions the thermal energy contacting element 20.

The thermal energy contacting element 20 comprises a thermal block 54 and a plurality of weighted slip pins 56. The weighted slip pins 56 are thermally interconnected to the thermal block 54 and are positioned in apertures of the thermal block 54. The weighted slip pins 56 are designed to contact the patient's venous plexus area in such a way that it minimizes the tissue interface pressure on the patient. In addition, the weighted slip pins are designed to conform to the shape of the patient's venous plexus area to maximize the contact of the thermal energy contacting element 20 to the venous plexus area.

The thermal block 54 could be a conventional heater block and/or cooling block.

To ensure the patient's venous plexus area contacts the weighted slip pins, the negative pressure, thermal energy device 10 can have an inflatable seal 14 positioned on the opposite side of the patient's body portion having the venous plexus area, as described in the first embodiment.

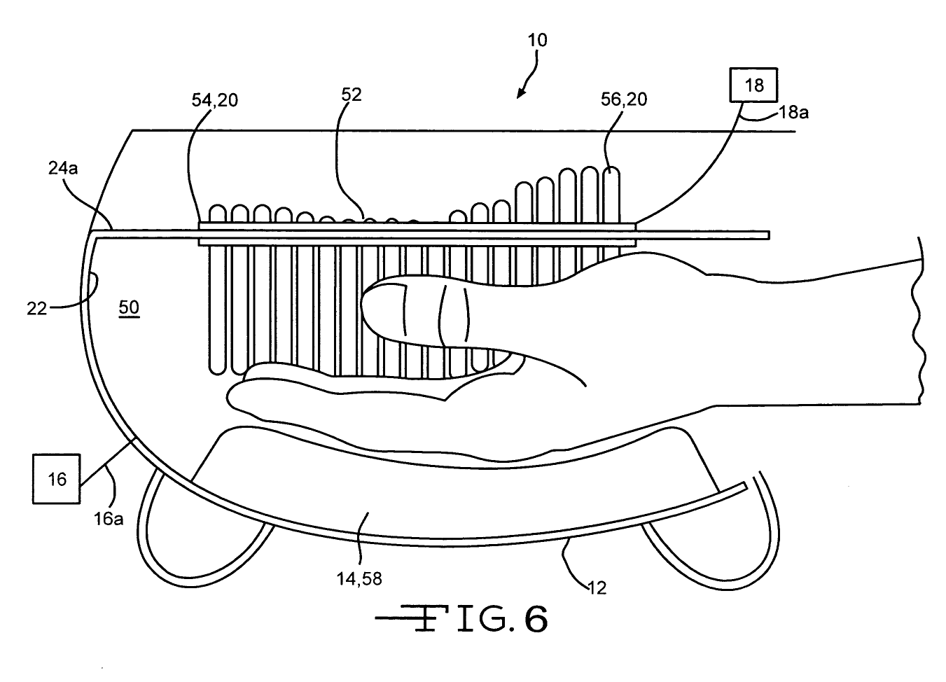

Alternatively, a cushioned foam 58 can be positioned on the opposite side of the patient's body portion having the venous plexus area to ensure the patient's body portion having a venous plexus area contacts the weighted slip pins. In this alternative embodiment any seal 14 (not shown in FIG. 6) described in the current section entitled detailed description of the invention except the first embodiment, and the prior art (though undesirable), can be used with this sixth embodiment.

The vacuum system 16 can be the conventional embodiments for this sixth embodiment.

### Seventh Embodiment

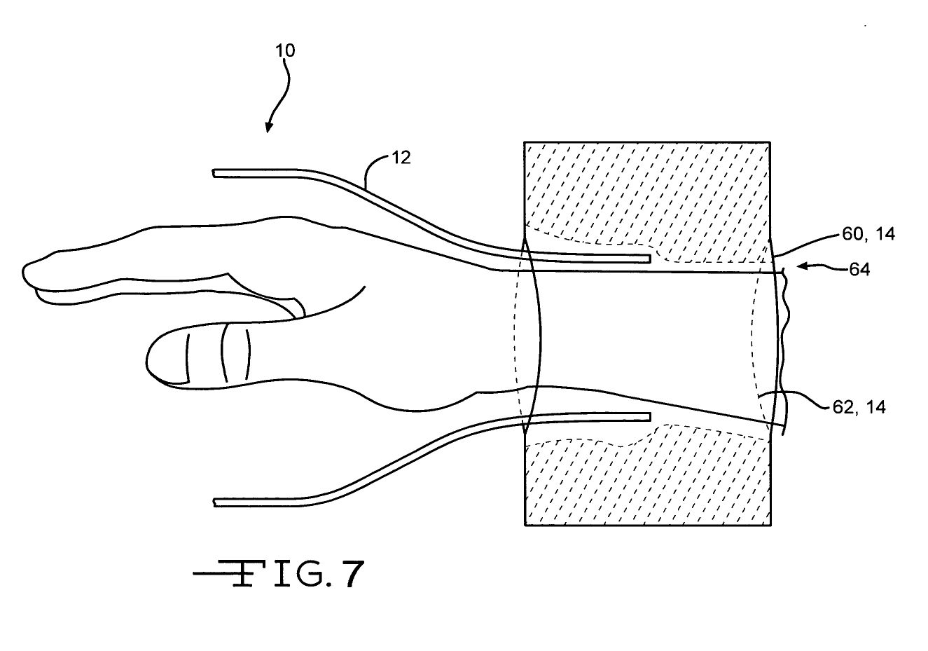

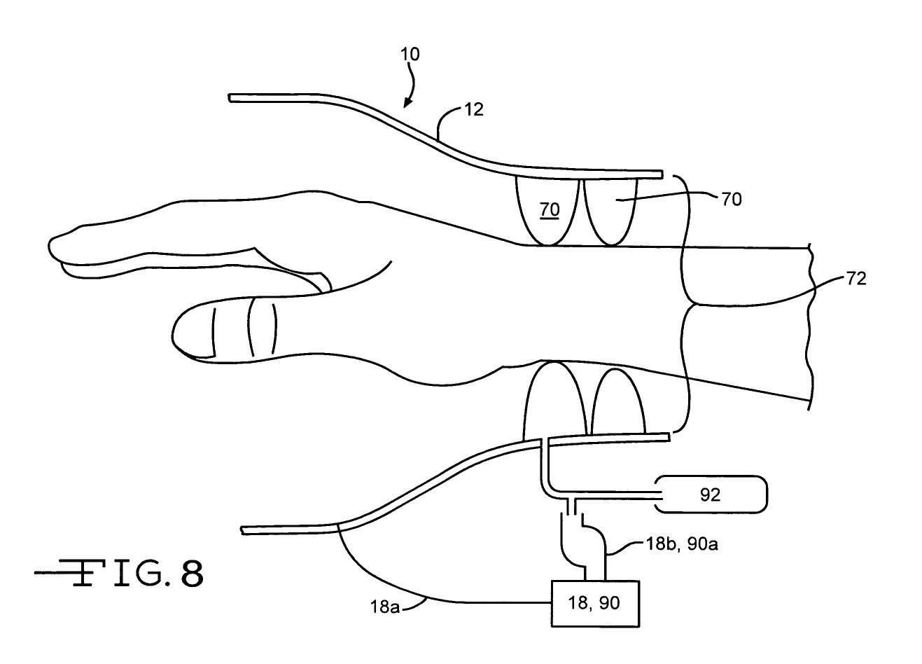

FIG. 7 illustrates a seventh embodiment of the current invention. This embodiment is directed exclusively to an embodiment of the seal 14. In this embodiment, the seal 14 comprises a first sheet 60 and a second sheet 62. The first sheet 60 and the second sheet 62 are materials that can be bonded, adhered to, static electrically connected to, and/or connected to each other. The first sheet 60 and the second sheet 62 can be the same materials, different materials, the same piece of material, or different pieces of materials. The first sheet 60 and the second sheet 62 can be a polymeric material, a polymeric metallic material, a metallic material and combinations thereof. The first sheet 60 and the second sheet 62 can contain conventional adhesives that are medically acceptable for contact to a patient's skin. The first sheet 60 and/or the second sheet 62 can have apertures and/or gaps 64 between the sheets 60, 62 that allow predetermined amounts of negative pressure to escape from the enclosure 12.

The seal 14 of the first and second sheets 60, 62 provide low interface pressure to the patient's body. This embodiment also allows medical intravenous lines to be inserted into the patient near the patient's body portion having the venous plexus area. Applying such lines were essentially impossible with the prior art's seals due to the difficulty of inserting and removing the patient's body portion from the prior art negative pressure, thermal energy device. If this embodiment is used, the first and second sheets 60, 62 should be sterile when and if an intravenous needle penetrates the sheets 60, 62 and the patient's skin.

The seventh embodiment can be incorporated with the prior art negative pressure, thermal energy devices and the embodiments of the present invention.

### Eighth Embodiment

The eighth embodiment is an alternative seal 14 embodiment. In this embodiment the seal 14 is an inflatable bladder 70 on the interior perimeter of the enclosure's 12 opening 72 that receives the patient's body portion. The inflatable bladder 70 is made of conventional bladder material used in association with hospital mattresses. An example of such bladder materials and corresponding pump 90 (with conduit 90a) are Gaymar's AirFlo mattress material and pump. There could also be a transducer 92 to ensure the proper pressure is applied to the inflatable bladder 70.

The inflatable bladder 70 receives a fluid at a predetermined pressure. Preferably the predetermined pressure in the inflatable bladder 70 is around 32 mmHg. There could also be a transducer 92 to ensure the proper pressure is applied to the inflatable bladder 70. The inflatable bladder 70 inflates to the predetermined pressure when the patient's body portion is properly positioned on the thermal energy contacting element 20. The predetermined pressure does not inhibit the blood flow through the venous plexus area but assists in the creation of the desired vasodilation that is required.

The fluid can be air, water, or any other fluid that can be provided to the inflatable bladder 70 at the predetermined pressure. In one embodiment the fluid can also be provided by Gaymar's Medi-Therm device that could be used with the thermal energy system 18. It is known that using two of Gaymar's Medi-Therm devices can deliver two fluids (same or different) having two different (or same) temperatures and two different (or same) pressures to two different locations at the same time.

The inflatable bladder 70 allows the negative pressure to leak at a controllable rate that does not create a tourniquet effect on the patient. The inflatable bladder 70 simultaneously applies some pressure to sections of the patient's body portion.

When the patient's body portion is to be removed from the negative pressure, thermal energy device 10, the inflatable bladder 70 is deflated (or partially deflated) to allow the patient's body portion to be removed with minimal effort.

This embodiment allows medical intravenous lines to be inserted into the patient near the patient's body portion having the venous plexus area. Applying such medical lines were essentially impossible with the prior art's seals due to the difficulty of inserting and removing the patient's body portion from the prior art negative pressure, thermal energy device.

The eighth embodiment can be incorporated with the prior art negative pressure, thermal energy devices and the embodiments of the present invention.

### Ninth Embodiment

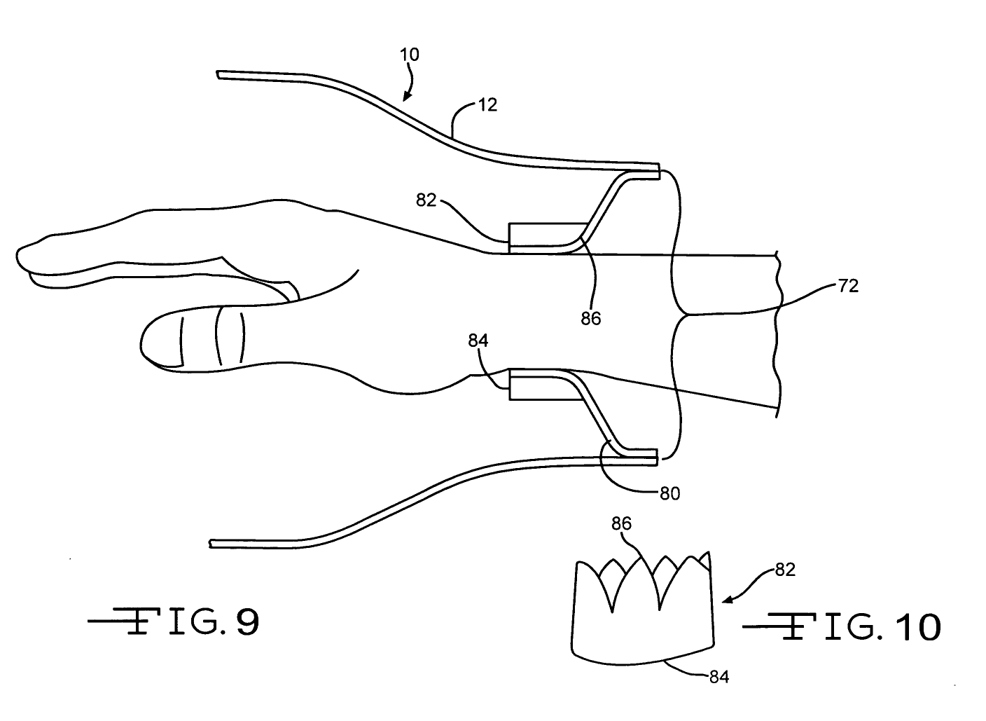

FIG. 9 illustrates another seal 14 embodiment. This embodiment entails a polymeric webbing material 80 (similar to the material used in the prior art) with a foam material 82 positioned between the webbing material 80 and the patient's body portion. The foam material 82 is preferably connected, attached, and/or adhered to the webbing material 80 in such a way that the webbing material does not contact the patient.

The foam material 82 is structured like a crown, see FIG. 10. The crown circumscribes the wrist and forearm of the patient. This crown structure means that the crown's points 86 (apexes) are directed toward the upper arm while the circulet portion 84 (base) of the crown seal along the wrist and forearm. The apexes and the base of the crown can also be reversed in position as illustrated in FIG. 10. The intent of the crown structure is that the seal will prevent the tendency of the arm and hand to be drawn further into the pressure vessel 12 upon the application of the negative pressure in the vessel 12.

The crown structure has a base 84 and apexes 86. The crown structure decreases the tissue interface pressure applied to the patient's body, which is desired, and simultaneously allows the negative pressure in the enclosure 12 to escape at a desired soft seal rate.

This embodiment allows medical intravenous lines to be inserted into the patient near the patient's body portion having the venous plexus area. Applying such lines were essentially impossible with the prior art's seals due to the difficulty of inserting and removing the patient's body portion from the prior art negative pressure, thermal energy device.

The ninth embodiment can be incorporated with the prior art negative pressure, thermal energy devices and the embodiments of the present invention.

### Alternative embodiments

The present invention can also have devices that monitor vasodilation, vasoconstriction, body core temperature, and/or apply compression therapy to other portions of the patient's body. These embodiments are disclosed in U.S. Pat. Nos. 5,683,438; 6,602,277; 6,673,099; 6,656,208; 6,966,922; 7,122,047; and 6,974,442; and U.S. patent application Ser. No. 11/588,583, filed on Oct. 27, 2006, which are hereby incorporated by reference.

It is appreciated that various modifications to the inventive concepts described herein may be apparent to those of ordinary skill in the art without departing from the scope of the present invention as defined by the herein appended claims.

We claim:

1. A thermal energy and negative pressure therapies device comprising (1) an enclosure having an opening to receive a portion of a patient's body that contains a venous plexus area, (2) a vacuum system that creates a negative pressure in the enclosure, (3) a soft seal to maintain the negative pressure in the enclosure, and (4) a thermal energy system having a thermal energy contacting element so the patient's venous plexus area contacts the thermal energy contacting element;
the thermal energy contacting element is selected from the group consisting of
(a) a metallic thermal energy transfer unit that receives a fluid and/or contains resistors when the soft seal has a bladder seal positioned in the interior of the enclosure and the metallic thermal energy transfer unit contacts the patient's venous plexus area and the bladder seal contacts the patient's body portion on the opposite side of the venous plexus area,
(b) a light source that radiates heat from within the enclosure toward the patient's venous plexus area;
(c) a cold source positioned within the enclosure and extracts thermal energy from the patient's venous plexus area;
(d) a convective fitting object positioned on the patient's venous plexus area,
(e) conductive beads extending from and interconnected to a conductive plate, and
(f) a thermal block in combination with a plurality of weighted slip pins.

2. The device of claim 1 wherein the soft seal is selected from the group consisting of a bladder seal positioned in the interior of the enclosure and the bladder seal and the thermal energy contacting element are on opposite sides of the patient's venous plexus area; an inflatable bladder positioned at or near the enclosure's opening; and a first sheet and a second sheet interconnected to each other with openings therein.

3. The device of claim 1 wherein when the thermal energy contacting element is the light source, the light source is selected from the group consisting of a light source that radiates heat having a narrow band of non-ionizing light between 500 to 2000 nm and a light source that radiates heat having a broad band of non-ionizing light between 500 to 2000 nm.

4. The device of claim 3 wherein the patient's body in the enclosure is covered with a form fitting material, and negative pressure is between the form fitting material and the patient's body.

5. The device of claim 3 wherein the enclosure has a mezzanine layer that separates the enclosure into a first section that receives the patient's venous plexus area and a second area that contains the thermal energy contacting element.

6. The device of claim 5 wherein the mezzanine layer has thermocouple devices interconnected to the thermal energy system to control the thermal energy contacting element.

7. The device of claim 4 wherein the form fitting material has thermocouple devices interconnected to the thermal energy system to control the thermal energy contacting element.

8. The device of claim 1 wherein the convective fitting object has negative pressure between the convective fitting object and the patient's body.

9. The device of claim 1 wherein the conductive plate provides thermal energy selected from the group consisting of thermal energy having a temperature (a) above the patient's body core temperature, and (b) below the patient's body core temperature.

10. The device of claim 1 wherein the thermal block provides thermal energy selected from the group consisting of thermal energy having temperature (a) above the patient's body core temperature, and (b) below the patient's body core temperature.

11. A thermal energy and negative pressure therapies device comprising an enclosure having an opening to receive a portion of a patient's body that contains a venous plexus area, (2) a vacuum system that creates a negative pressure in the enclosure, (3) a soft seal to maintain the negative pressure in the enclosure, and (4) a thermal energy system having a thermal energy contacting element so the patient's venous plexus area contacts the thermal energy contacting element;
the soft seal is selected from the group consisting of a bladder seal positioned in the interior of the enclosure and the bladder seal and the thermal energy contacting element are on opposite sides of the patient's venous plexus area; an inflatable bladder positioned at or near the enclosure's opening; and a first sheet and a second sheet interconnected to each other with openings therein.

12. The device of claim 11 wherein the thermal energy contacting element is selected from the group consisting of
(a) a metallic thermal energy transfer unit that receives a fluid and/or contains resistors when the soft seal has the bladder seal positioned in the interior of the enclosure and the metallic thermal energy transfer unit contacts the patient's venous plexus area and the bladder seal contacts the patient's body portion on the opposite side of the venous plexus area,
(b) a light source that radiates heat from within the enclosure toward the patient's venous plexus area;
(c) a cold source positioned within the enclosure and extracts thermal energy from the patient's venous plexus area;
(d) a convective fitting object positioned on the patient's venous plexus area,
(e) conductive beads extending from and interconnected to a conductive plate, and
(f) a thermal block in combination with a plurality of weighted slip pins.

13. The device of claim 12 wherein when the thermal energy contacting element is the light source, the light source is selected from the group consisting of a light source that radiates heat having a narrow band of non-ionizing light between 500 to 2000 nm and a light source that radiates heat having a broad band of non-ionizing light between 500 to 2000 nm.

14. The device of claim 13 wherein the patient's body in the enclosure is covered with a form fitting material, and negative pressure is between the form fitting material and the patient's body.

15. The device of claim 13 wherein the enclosure has a mezzanine layer that separates the enclosure into a first section that receives the patient's venous plexus area and a second area that contains the thermal energy contacting element.

16. The device of claim 15 wherein the mezzanine layer has thermocouple devices interconnected to the thermal energy system to control the thermal energy contacting element.

17. The device of claim 14 wherein the form fitting material has thermocouple devices interconnected to the thermal energy system to control the thermal energy contacting element.

18. The device of claim 12 wherein the convective fitting object has negative pressure between the convective fitting object and the patient's body.

19. The device of claim 12 wherein the conductive plate provides thermal energy selected from the group consisting of thermal energy having a temperature (a) above the patient's body core temperature, and (b) below the patient's body core temperature.

20. The device of claim 12 wherein the thermal block provides thermal energy selected from the group consisting of thermal energy having a temperature (a) above the patient's body core temperature, and (b) below the patient's body core temperature.
